SEQUENCE LISTING (10 sequences)

SEQ ID NO: 1 - RNA, 33 nt, Drosophila melanogaster
uggcggagguggguaucuggauggguuggcucg

SEQ ID NO: 2 - RNA, 21 nt, Drosophila melanogaster
cucacauuuacauauucacag

SEQ ID NO: 3 - RNA, 20 nt, Drosophila melanogaster
guggguaucuggaugugguu

SEQ ID NO: 4 - RNA, 33 nt, Homo sapiens
ugucauaagccaugccuucugcaauuugccucg

SEQ ID NO: 5 - DNA, 22 bp, Homo sapiens
caaauugcagaaggcauggctt

SEQ ID NO: 6 - RNA, 36 nt, Homo sapiens
augacauaagguggaagccgggcauaacuuaguaaa

SEQ ID NO: 7 - DNA, 21 bp, Homo sapiens
guuaugcccggcuuccacctt

SEQ ID NO: 8 - DNA, 21 bp, Homo sapiens
gguggaagccgggcauaactt

SEQ ID NO: 9 - RNA, 36 nt, Homo sapiens; identical to SEQ ID NO: 6

SEQ ID NO: 10 - RNA, 27 nt, Homo sapiens
augcccggcuuccaccuuaugucauag

(19) United States
(12) Patent Application Publication
(10) Pub. No.: US 2015/0275206 A1
(43) Pub. Date: Oct. 1, 2015

(54) POLYMER CONJUGATES FOR DELIVERY OF BIOLOGICALLY ACTIVE AGENTS

(71) Applicant: CARNEGIE MELLON UNIVERSITY, PITTSBURGH, PA (US)

(72) Inventors: Subha Ranjan Das, Pittsburgh, PA (US); Eduardo Paredes, Cincinnati, OH (US); Saadyah Averick, Pittsburgh, PA (US); Krzysztof Matyjaszewski, Pittsburgh, PA (US)

(73) Assignee: CARNEGIE MELLON UNIVERSITY, PITTSBURGH, PA (US)

(21) Appl. No.: 14/418,987

(22) PCT Filed: Aug. 1, 2013
(86) PCT No.: PCT/US2013/053314

§ 371 Date: Feb. 2, 2015

Related U.S. Application Data
(60) Provisional application No. 61/742,097, filed on Aug. 2, 2012.

(51) Int. Cl. C12N 15/113

(52) U.S. Cl. CPC C12N 15/113; C12N 2310/14; C12N 2310/351; C12N 2320/51; C12N 2320/32

(57) ABSTRACT

A delivery system for delivering oligonucleotides includes a conjugate includes a complexing agent including an oligonucleotide, a peptide nucleic acid or chimera thereof and a polymer covalently attached to the complexing agent. The complexing agent of the conjugate is adapted to complex a biologically active agent thereto after formation of the conjugate.

### CROSS-REFERENCE TO RELATED APPLICATIONS

This application claims benefit of U.S. Provisional Patent Application Ser. No. 61/742,097, filed Aug. 2, 2012, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Oligonucleotides such as DNA and/or RNA have been used for the treatment of a number of conditions. Synthetic DNA and modified DNA sequences can, for example, regulate cellular target sequences in anti-gene or anti-sense applications. Further, the process of RNA interference (RNAi), for example, has altered the landscape of both basic research to examine gene function pathways and therapeutic paradigms. RNAi may be initiated by delivery of exogenous short interfering RNA (siRNA) to cells. These are typically delivered in short 21-23 mer duplexes and other forms that are processed by the cellular machinery. The duplexes interact with the cellular RNA-induced silencing complex (RISC) that eventually uses one strand from the duplex, termed the guide or antisense strand, to silence a target mRNA. Barriers to using exogenous short interfering RNA duplexes are their susceptibility to degradation and their cell impermeability. Although chemical modifications can overcome the lability of the native sugar-phosphate backbone towards hydrolysis and nucleases, cell permeability still presents a significant challenge.

Delivery of exogenous oligonucleotides has therefore required attachment of delivery agents such as lipids or peptides or complexation with transfection reagents that enhance cell permeability and provide additional protection of the RNA duplex from nuclease degradation. Non-viral transfection reagents have relied on formation of a non-specific polyplex between cationic lipid nanoparticles or polymers and the anionic siRNA. Although widely studied for siRNA delivery, these materials have several practical limitations such as relying on ionic interactions to prepare the polyplex which can be destabilized during circulation or in media.

Alternative methods for siRNA delivery rely on direct covalent modifications of the 5′- and/or 3′-terminus of an siRNA duplex with lipid groups, small molecules such as biotin and folate, peptides, nanoparticles, carbon nanotubes, nanostructured DNA or poly(ethylene glycol) (PEG). Modification of siRNA with linear PEG or brush PEG, has been accomplished using disulfide formation or a Michael-type addition between a thiol and maleimide group. While the disulfide linkage allows for release of the siRNA duplex following cellular internalization, the generation of redox sensitive thiols and disulfides that can undergo undesired side reactions or premature degradation poses challenges in synthesis and purification of the polymer-siRNA conjugates. While such siRNA-polymer conjugates have enhanced nuclease stability, some require additional transfection agent, limiting their overall utility as a stand-alone delivery system.

### SUMMARY

In one aspect, a delivery system for delivering a biologically active agent includes a conjugate including a complexing agent including an oligonucleotide, a peptide nucleic acid or chimera thereof and a polymer covalently attached to the complexing agent. The complexing agent of the conjugate is adapted to complex a biologically active agent thereto after formation of the conjugate. The polymer may, for example, be attached to the complexing agent at a terminus of the complexing agent or at a position internal to the complexing agent (that is, between the ends or termini of the complexing agent). The delivery system may, for example, include at least a second polymer covalently attached to the complexing agent. The second polymer (or further polymers) may, for example, be attached to the complexing agent at a terminus of the complexing agent or at a position internal to the complexing agent.

The biologically active agent may, for example, be a partially or fully complementary strand of RNA, DNA, PNA or chimera. In a number of embodiments, the biologically active agent is a partially or fully complementary strand of guide RNA. The partially or fully complementary strand of guide RNA may, for example, be adapted to effect RNA interference. In a number of embodiments, the complexing agent includes a passenger strand of RNA.

The conjugate may, for example, be prepared by reacting a functional group on the polymer(s) with a functional group or groups on the oligonucleotide, peptide nucleic acid or chimera. In a number of embodiments, the reaction of the functional group on the polymer to with the functional group on the oligonucleotide is a “click” reaction. The reaction of the functional group on the polymer to with the functional group on the oligonucleotide may, for example, be a Staudinger ligation, an azide-alkyne cycloaddition, a reaction of tetrazine with a trans-cyclooctene, a disulfide linking reaction, a thiol ene reaction, a hydrazine-aldehyde reaction, a hydrazine-ketone reaction, a hydroxyl amine-aldehyde reaction, a hydroxyl amine-ketone reaction or a Diels-Alder reaction.

In a number of embodiments, the polymer(s) is/are formed via controlled radical polymerization. The polymer(s) may, for example, be formed via atom transfer radical polymerization or activators generated by electron transfer atom transfer radical polymerization.

In a number of embodiments, the polymer or polymers have a molecular weight between approximately 1 kDa and 60 kDa or between approximately 1 kDa and 50 kDa. The polymer(s) may, for example, have a polydispersity between 1 and 2, between 1 and 1.5 or between 1 and 1.2.

In a number of embodiments, the polymer or polymers is/are independently a polyacrylate, a polymethacrylate, a polyacrylamide, a polymethacrylamide, a polystyrene, a polyethylene oxide, a poly(organo)phosphazene, a poly-1-lysine, a polyethyleneimine, a poly-d,l-lactide-co-glycolide, or a poly(alkylcyanoacrylate).

The polymer(s) may include at least one of a targeting agent group or a group that is cationic under physiological conditions such that the conjugate is auto-transfecting.

In a number of embodiments, the biologically active agent separates from the conjugate in vivo.

In another aspect, a method of synthesizing a delivery system for delivering a biologically active agent includes preparing a conjugate by reacting a polymer with complexing agent to covalently attach the polymer to the complexing agent. The complexing agent includes an oligonucleotide, a peptide nucleic acid or chimera thereof. The conjugate is adapted to complex a biologically active agent thereto after preparation of the conjugate. The method further includes complexing the biologically active agent to the conjugate. The method may, for example, include covalently attaching at least a second polymer to the complexing agent. The polymer(s) may, for example, be attached to the complexing agent at a terminus of the complexing agent or at a position internal to the complexing agent.

In a number of embodiments, the biologically active agent is a partially or fully complementary strand of RNA, DNA, PNA or chimera. In a number of embodiments, the biologically active agent is a partially or fully complementary strand of guide RNA. The partially or fully complementary strand of guide RNA may, for example, be adapted to effect RNA interference. In a number of embodiment, the oligonucleotide of the complexing agent includes a passenger strand of RNA.

The conjugate may be prepared by reacting a functional group on the polymer(s) with a functional group or groups on the oligonucleotide, PNA or chimera. In a number of embodiments, the reaction of the functional group on the polymer to with the functional group on the oligonucleotide is a “click” reaction. The reaction of the functional group on the polymer(s) with the functional group on the oligonucleotide may, for example, be a Staudinger ligation, an azide-alkyne cycloaddition, a reaction of tetrazine with a trans-cyclooctene, a disulfide linking reaction, a thiol ene reaction, a hydrazine-aldehyde reaction, a hydrazine-ketone reaction, a hydroxyl amine-aldehyde reaction, a hydroxyl amine-ketone reaction or a Diels-Alder reaction.

In a number of embodiments, the polymer(s) is/are formed via controlled radical polymerization. The polymer(s) may, for example, be formed via atom transfer radical polymerization or activators generated by electron transfer atom transfer radical polymerization.

In a number of embodiments, the polymer or polymers have a molecular weight between approximately 1 kDa and 60 kDa or between approximately 1 kDa and 50 kDa. The polymer(s) may, for example, have a polydispersity between 1 and 2, between 1 and 1.5 or between 1 and 1.2.

In a number of embodiments, the polymer or polymers is/are independently a polyacrylate, a polymethacrylate, a polyacrylamide, a polymethacrylamide, a polystyrene, a polyethylene oxide, a poly(organo)phosphazene, a poly-1-lysine, a polyethyleneimine, a poly-d,l-lactide-co-glycolide, or a poly(alkylcyanoacrylate).

The polymer(s) may include at least one of a targeting agent group or a group that is cationic under physiological conditions such that the conjugate is auto-transfecting.

In a number of embodiments, the biologically active agent separates from the conjugate in vivo.

In a further aspect, an auto-transfecting system includes a conjugate including a complexing agent including an oligonucleotide, a peptide nucleic acid or chimera and at least one polymer covalently attached to the complexing agent. The polymer or polymers may, for example, be attached to the complexing agent at a terminus of the complexing agent or at a position internal to the complexing agent. Each of the polymers has a molecular weight distribution less than 2 and a molecular weight between 1 and 60 kDa. At least one of the polymers further includes at least one of a targeting agent group or a group that is cationic under physiological conditions. The system further includes a biologically active agent complexed to the complexing agent of the conjugate after formation of the conjugate. The biologically active agent may, for example, include a partially or fully complementary strand of RNA, DNA, PNA or chimera thereof. In a number of embodiments, the biologically active agent is a partially or fully complementary strand of guide RNA, and the partially or fully complementary strand of guide RNA is adapted to effect RNA interference.

The present systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates an embodiment of a method for synthesis of representative escorted duplex siRNA conjugates or siRNA delivery systems hereof.

FIG. 1B illustrates the chemical structure of polymer P^{M}.

FIG. 1C illustrates the chemical structure of polymer P^{T}.

FIG. 1D illustrates the chemical structure of polymer P^{N}.

FIG. 1E illustrates a polyacrylamide gel electrophoresis study of the representative siRNA, three P^{x}Ep-siRNA systems and polymer P^{N} alone.

FIG. 2 illustrates a study of the nuclease stability of the representative P^{x}Ep-siRNA (x=M, T, N) delivery systems compared to unmodified siRNA, wherein samples were incubated with (+) and without (−) RNase A for 2 hours and run on a non-denaturing polyacrylamide gel and stained with EtBr.

FIG. 3 illustrates Dicer cleavage of the P^{N}Ep-siRNA system and related constructs.

FIG. 4A illustrates a schematic representation of internalization and RNAi induction.

FIG. 4B illustrates a graph of relative Renilla luciferase (Rluc) signal for cells only. siRNA, siRNA+FUGENE HD, and P^{x}Ep-siRNA (wherein x=M, T, N; at 50, 125 and 250 nM).

FIG. 5A illustrates a Western blot analysis of the inhibition of Lck in Hek293 cells using an auto-transfecting siRNA.

FIG. 5B illustrates a graph showing densitometric quantitation of Western blots with the Lck signal normalized to the actin control.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a polymer” includes a plurality of such polymers and equivalents thereof known to those skilled in the art, and so forth, and reference to “the polymer” is a reference to one or more such polymers and equivalents thereof known to those skilled in the art, and so forth.

In a number of embodiments hereof, delivery systems for delivering biologically active agents include a complexing agent (for example, an oligonucleotide, a peptide nucleic acid (PNA) or chimera thereof) and a polymer covalently attached to the complexing agent (for example, at least one terminus of the complexing agent or at an internal position on the complexing agent). The complexing agent of the conjugate is adapted to complex a partially or fully complementary oligonucleotide strand (for example. RNA, DNA or chimera), a PNA strand, or chimera having 10 to 40 residues. In a number of embodiments, conjugate delivery systems are synthesized by reacting a polymer with a complexing agent to covalently attach the polymer to the complexing agent to at least one terminus of the complexing agent or to an internal position thereon. As described above, the complexing agent includes an oligonucleotide, a peptide nucleic acid or chimera adapted to complex a complementary or partially complementary oligonucleotide, PNA strand, or a chimeric strand. The complementary or partially complementary strand may, for example, be complexed to complexing agent of the conjugate after formation of the conjugate.

The complexing agent-polymer conjugates or constructs may, for example, serve as stand-alone RNA, DNA and/or PNA delivery vehicles or systems. In that regard, in a number of embodiment, the delivery systems hereof are auto-transfecting (that is, no additional transfection agent is required).

A complexing agent such as an oligonucleotide (for example, RNA, DNA, or chimera), a PNA, or an oligonucleotide/PNA chimera may, for example, be conjugated to a polymer on either or both ends of the complexing agent Alternatively, a complexing agent may be conjugated to one or more polymers at a position or positions between the ends of the complexing agent. In a number of embodiments, the complexed biologically active agent (for example, a fully or partially complementary strand of RNA or DNA) is released from the conjugate in vivo and is bioactive as a single strand. In such embodiments, the complexing agent conjugated to the polymer need not be a complementary strand of passenger RNA or DNA, but can be another partially or fully complementary oligonucleotide, PNA or chimera. DNA may, for example, in certain circumstance be less expensive to synthesize than RNA and may be used as a complexing agent to complex a complementary strand or RNA.

As used herein an oligonucleotide molecule is a biopolymer composed of 10 or more nucleotide monomers covalently bonded in a chain such as RNA, DNA and or a peptide nucleic acid. A peptide nucleic acid (PNA) is an artificially synthesized polymer similar to DNA or RNA. However, whereas DNA and RNA include a deoxyribose and ribose sugar backbone, respectively, the backbone of PNA include repeating N-(2-aminoethyl)-glycine units linked by peptide bonds. Chimera (or chimeric strands) include units of DNA, RNA and/or PNA.

In a number of illustrative or representative embodiments, the complexing agent is the sense or passenger strand (sometimes referred to herein as p-RNA) of siRNA. The delivery system may, for example, further include a complementary antisense or guide strand (sometimes referred to herein as g-RNA) complexed to the p-RNA. In a number of embodiments, the g-RNA is released from the conjugate in vivo and is bioactive (for example, to effect and RNAi response) as a single strand. As described above, in such embodiments, the oligonucleotide conjugated to the polymer need not be a complementary strand of passenger RNA, but can be another complementary oligonucleotide such as DNA or a PNA.

As described above, in a number of embodiments, RNA or DNA (for example a guide strand or g-RNA of siRNA) is hybridized/complexed to the complexing agent-polymer conjugate after formation of the conjugate. In such embodiments, damage of the RNA or DNA resulting from the conjugation reaction conditions may, for example, be avoided. In other embodiments, the polymer or polymers may be conjugated to a complex/duplex including the RNA or DNA.

A suitable polymer or polymers directly conjugated to only the complexing agent (for example, to only the passenger strand of an siRNA complex) confers both desirable properties of nuclease resistance and cell permeability to the entire complex/duplex. The complexing agent-polymer conjugates hereof reduce or eliminate many of the problems associated with existing delivery systems such as siRNA delivery systems. In embodiments in which a delivery system hereof is used to deliver a strand of g-RNA. The stabilized and auto- or self-transfecting delivery system permits the guide strand to, for example, effectuate an RNAi response.

Many different types of polymers may be conjugated to the complexing agents hereof. The polymers may, for example, be homopolymers, block copolymers, linear copolymers, block copolymers or triblock copolymers including a random copolymer segments. In a number of embodiments, the molecular weight of the polymer is between approximately 1 kDa and 60 kDa or between approximately 1 kDa and 50 kDa. Controlling the polydispersity of the polymer may, for example, be important to ensure desired and controlled polymer properties as well as adequate renal excretion of the delivery systems hereof (or of degradation products thereof). In that regard, maintaining molecular weight of the polymers no greater than 60 kDa or 50 kDa may assist in ensuring adequate renal excretion. Polymers used in forming the conjugates hereof may have a polydispersity index or PDI of less than 2.0, less than 1.5, less than 1.3, or even less than 1.2. The PDI is defined by the ratio of the weight average molecular weight to the number average molecular weight, M_{w}/Mₙ.

In a number of embodiments, a polymer or polymers of the conjugate include at least one of a targeting agent group or a group that is cationic under physiological conditions. Such targeting groups and/or cationic groups assist in effecting auto-transfection. Cationic group may be inherently cationic (such as, for example, a quaternary ammonium group, a phosphonium group or a sulfonium group). Alternatively, cationic group may become cationic under physiological conditions (for example, an amine group that becomes protonated under physiological conditions).

Targeting groups or agents are groups or moieties used for targeting the polymer or delivery systems hereof, for example, to cells, to specific cells, to tissues or to specific locations in a cell. Targeting groups enhance the association of molecules with a cell or other specific location. Examples of targeting groups include those that target to the asialoglycoprotein receptor by using asialoglycoproteins or galactose residues. Other proteins such as insulin, EGF, or transferrin, for example, may be used for targeting. Other targeting groups include molecules that interact with membranes such as fatty acids, cholesterol, dansyl compounds, and amphotericin derivatives. A variety of ligands have been used to target drugs and genes to cells and to specific cellular receptors. The ligand may, for example, seek a target within the cell membrane, on the cell membrane or near a cell. Binding of a ligand to a receptor may, for example, initiate endocytosis

Polymer functionality may, for example, be linear or branched, and may include polyethylene glycol, a PEG-like group, amine bearing groups (including primary, secondary, tertiary amine groups), cationic groups (which may generally be any cationic group—examples include quaternary ammonium group, phosphonium group or sulfonium group), reactive groups for modification of polymer with, for example, small molecules (including, for example, dyes and targeting agents), polymers and biomolecules. Examples of suitable polymers include, but are not limited to, polyacrylate, polymethacrylates, plyacrylamides, polymethacrylamides, polystyrenes, polyethylene oxides (PEO), poly(organo)phosphazenes, poly-1-lysine, polyethyleneimine (PEI), poly-d,l-lactide-co-glycolide (PLGA), and poly(alkylcyanoacrylate).

In a number of representative embodiments, polymers were prepared with a functional group reactive with a functional group on at least one terminus of the complexing agent of the conjugate. As described above, a functional group on the polymer may also be reactive with an internal functional group of the complexing agent (that is, positioned between the ends or termini of the complexing agent). A polymer or polymers may also be prepared with a functional group internal to the polymer (that is, between the ends thereof) that is reactive with a functional group of the complexing agent. The resultant linking group(s) formed between the polymer and the complexing agent may be stable or degradable/cleavable. The linking group may be sensitive to, for example, local cellular environments. The linking group may, for example, be enzymatically cleavable. For example, short peptide or sugar sequences can be selectively cleaved by enzymes. The linking group may alternatively be pH sensitive (such as an acetal group or an oximine group) or redox sensitive (for example, a disulfide group). A cleavable linking group may be readily chosen to cleave relatively quickly upon reaching a target site such that the conjugate/biologically active agent complex is released or to cleave after the complexed bioactive RNA, DNA, PNA or chimera is released from the complexing agent-polymer conjugate.

In a number of representative embodiments hereof, one or more polymers were conjugated to p-RNA (as a complexing agent for g-RNA) using any reaction described as “click” reactions as, for example, described in U.S. Pat. No. 7,795,355 and/or Canalle, L., et al., “Polypeptide-polymer Bioconjugates, Chemical Society Reviews 39(1), 329-353 (2010), the disclosures of which are incorporated herein by reference. Such click reaction are suitable for reaction of other complexing agents hereof with one or more polymers. In general, “click” reactions are a group of high-yield chemical reactions that were collectively termed “click chemistry” reactions by Sharpless in a review of several small molecule click chemistry reactions. Kolb, H. C.; Finn, M. G.; Sharpless, K. B. Angew. Chemie, Interl. Ed. 40, 2004-2021 (2001), the disclosure of which is incorporated herein by reference. As used herein, a “click reaction” refers to a reliable, high-yield, and selective reaction having a thermodynamic driving force of greater than or equal to 20 kcal/mol. Click chemistry reactions may, for example, be used for synthesis of molecules comprising heteroatom links. One of the most frequently used click chemistry reactions involves cycloaddition between azides and alkynyl/alkynes to form the linkage comprising a substituted or unsubstituted 1,2,3-triazole. Certain click reactions may, for example, be performed in alcohol/water mixtures or in the absence of solvents and the products can be isolated in substantially quantitative yield.

Examples of suitable click reactions for use herein include, but are not limited to, Staudinger ligation, azide-alkyne cycloaddition (either strain promoted or copper(I) catalyzed), reaction of tetrazine with trans-cyclooctenes, disulfide linking reactions, thiolene reactions, hydrazine-aldehyde reactions, hydrazine-ketone reactions, hydroxyl amine-aldehyde reactions, hydroxyl amine-ketone reactions and Diels-Alder reactions. In such click reactions, one of the functional groups of the click reaction is on the complexing agent and the other of the functional groups of the click reaction is on the polymer. In a number of representative studies, p-RNA were prepared with azido groups that may be clicked with an alkyne moiety (which may or may not bear a cleavable linking group spacer with the polymer). Alternatively, p-RNA may be prepared with an alkyne group that may be clicked with an azido moiety of the polymer.

Polymers suitable for use herein may, for example, be prepared via anionic polymerization, cationic polymerization, condensation polymerization, free radical polymerization and controlled radical polymerization. Controlled radical polymerization (“CRP”) processes have been described by a number of workers. See, for example, Matyjaszewski, K., Ed. Controlled Radical Polymerization; ACS: Washington, D. C., 1998; ACS Symposium Series 685. Matyjaszewski, K., Ed. Controlled/Living Radical Polymerization. Progress in ATRP, NMP, and RAFT; ACS: Washington, D. C., 2000; ACS Symposium Series 768. Matyjaszewski, K., Davis, T. P., Eds. Handbook of Radical Polymerization; Wiley: Hoboken, 2002. Qiu, J.; Charleux, B.; Matyjaszewski, K. Prog. Polym. Sci. 2001, 26, 2083. Davis, K. A.; Matyjaszewski, K. Adv. Polym. Sci. 2002, 159, 1; Chemical Reviews (2001) 101, 2921-2990. The use of a CRP for the preparation of an oligo/polymeric material allows control over the molecular weight, molecular weight distribution of the (co)polymer, topology, composition and functionality of a polymeric material. The topology can be controlled, allowing the preparation of linear, star, graft or brush copolymers, formation of networks or dendritic or hyperbranched materials. Composition can be controlled to allow preparation of homopolymers, periodic copolymers, block copolymers, random copolymers, statistical copolymers, gradient copolymers, and graft copolymers. In a gradient copolymer, the gradient of compositional change of one or more comonomers units along a polymer segment can be controlled by controlling the instantaneous concentration of the monomer units in the copolymerization medium, for example. Molecular weight control is provided by a process having a substantially linear growth in molecular weight of the polymer with monomer conversion accompanied by essentially linear semilogarithmic kinetic plots for chain growth, in spite of any occurring terminations. Polymers from controlled polymerization processes typically have molecular weight distributions, characterized by the polydispersity index of less than or equal to 2. Polymers produced by controlled polymerization processes may also have a PDI of less than 1.5, less than 1.3, or even less than 1.2.

In CRP, further functionality may be readily placed on the oligo/polymer structure including side-functional groups, end-functional groups or can comprise site specific functional groups, or multifunctional groups distributed as desired within the structure. The functionality can be dispersed functionality or can comprise functional segments. The composition of the polymer may comprise a wide range of radically (co)polymerizable monomers, thereby allowing the properties of the polymer to be tailored to the application. Materials prepared by other processes can be incorporated into the final structure.

In general, polymerization processes performed under controlled polymerization conditions achieve the above-described properties by consuming the initiator early in the polymerization process and, in at least one embodiment of controlled polymerization, an exchange between an active growing chain and dormant polymer chain that is equivalent to or faster than the propagation of the polymer. In general, CRP process is a process performed under controlled polymerization conditions with a chain growth process by a radical mechanism, such as, but not limited to; ATRP, stable free radical polymerization (SFRP), specifically, nitroxide mediated polymerization (NMP), reversible addition-fragmentation transfer (RAFT), degenerative transfer (DT), and catalytic chain transfer (CCT) radical systems. A feature of controlled radical polymerizations is the existence of equilibrium between active and dormant species. The exchange between the active and dormant species provides a slow chain growth relative to conventional radical polymerization, all polymer chains grow at the same rate, although overall rate of conversion can be comparable since often many more chains are growing. Typically, the concentration of radicals is maintained low enough to minimize termination reactions. This exchange, under appropriate conditions, also allows the quantitative initiation early in the process necessary for synthesizing polymers with special architecture and functionality. CRP processes may not eliminate the chain-breaking reactions; however, the fraction of chain-breaking reactions is significantly reduced from conventional polymerization processes and may comprise only 1-10% of all chains.

ATRP is one of the most robust CRP and a large number of monomers can be polymerized providing compositionally homogeneous well-defined polymers having predictable molecular weights, narrow polydispersity, and high degree of end-functionalization. Matyjaszewski and coworkers disclosed ATRP, and a number of improvements in the basic ATRP process, in a number of patents and patent applications. See, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,627,314; 6,790,919; 7,019,082; 7,049,373; 7,064,166; 7,157,530 and U.S. patent application Ser. No. 09/534,827; PCT/US04/09905; PCT/US05/007,264; PCT/US05/007,265; PCT/US06/33152 and PCT/US2006/048656, the disclosures of which are herein incorporated by reference.

In a number of representative embodiments hereof, well defined polymers, with functional chain-ends (for example, azido chain ends), were prepared via ATRP as an exemplary controlled radical polymerization procedure. In a number of studies, activators generated by electron transfer atom transfer radical polymerization (AGET ATRP) was utilized to prepare a series of polymers grown from an initiator containing an azido functionality. Jakubowski, W.; Matyjaszewski, K., Macromolecules, 38, 4139-4146 (2005), the disclosure of which is incorporated herein by reference. This procedure is more biocompatible that a single step polymerization/coupling and would not be as likely to interact in a negative manner with the functionalized RNA of the representative embodiments.

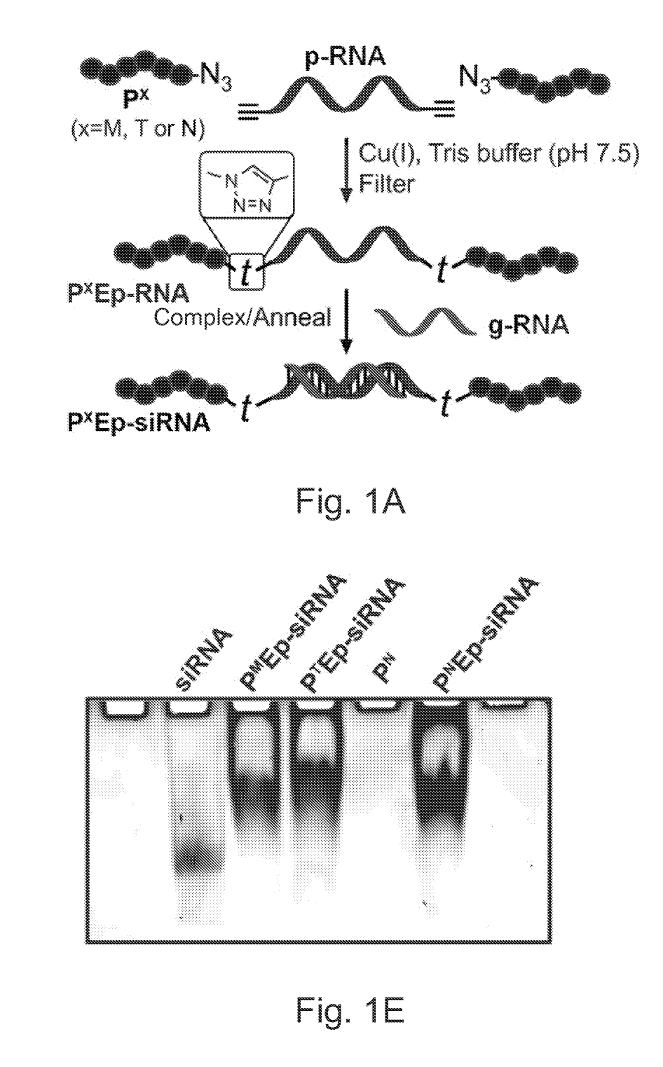

In a number of representative examples hereof, a copper catalyzed azide-alkyne cycloaddition (CuAAC) reaction was used as an efficient method for conjugating the RNA. The resultant triazole linkage is biocompatible. In a number of embodiments, copper catalyzed azide-alkyne cycloaddition click chemistry was used for efficient conjugation of polymers to one or both of the 5′- and 3′-termini of an RNA as illustrated in FIG. 1. In such embodiments, an extended RNA sequence that included the sense or passenger strand of an siRNA duplex (p-RNA) was synthesized with alkyne groups at one or both termini using standard commercially available reagents. The purified RNA was click-conjugated using CuAAC conditions that are compatible with naïve RNA (i.e., unprotected and with free 2′-hydroxyl groups).

In several studies hereof, a series of representative, well-defined azide-terminated polymers for click-conjugation to the bis-alkyne p-RNA, were synthesized via AGET ATRP. In that regard, three biocompatible polymers were synthesized to probe their ability to confer both nuclease resistance and cell permeability to siRNA. The polymers were: a PEG-methacrylate-pOEOMA₄₇₅ (P^{M}), a temperature responsive copolymer, pOEOMA₃₀₀-co-MEO₂MA (P^{T}) that is more hydrophobic than P^{M} (lower critical solution temperature for P^{T} in water is ca. 39° C.) and a copolymer containing amino groups that can be cationic at neutral pH, pOEOMA₄₇₅-co-DMAEMA (P^{N}). These monoazido-functional polymers all had a molecular weight of Mₙ˜21000 and a narrow molecular weight distribution, M_{w}/Mₙ<1.2. Such polymers have favorable cytocompatible properties.

These azido-terminated polymers and bis-alkyne terminated p-RNA were conjugated under suitable conditions suitable for oligonucleotide click-reactions. A twenty-fold molar excess of azido-polymer to RNA was used to ensure click-conjugation of both termini without RNA degradation. As illustrated in FIG. 1A, the reaction was in Tris buffer (pH 7.5) with 0.6% acetonitrile as minor co-solvent without any additional Cu(I) stabilizing ligand. The p-RNA was conjugated to monoazido-functionalized polymers P^{x} (x=M, T, N; wherein P^{M}=POEOMA475, P^{T}=POEOMA300-co-MEO₂MA, P^{N}=POEOMA475-co-DMAEMA) and purified by a simple filtration step. The structures of the monoazido-functionalized polymers are provided in FIGS. 1B through 1D. Subsequently a strand of g-RNA was annealed to the conjugate to formed a polymer “escort” duplex (or siRNA delivery system), P^{x}Ep-siRNA (wherein, x=M, T, N). Following a ninety minute reaction, a simple purification step using a centrifugal filter device with a 30000 molecular weight cutoff (MWCO) removes catalyst and excess unreacted polymers and provides the P^{x}Ep-RNAs (see FIG. 1A). This procedure is the first instance of click-conjugation of polymer to RNA. Following the click-conjugation of the polymers to, for example, both termini of p-RNA, the complementary RNAi competent 21-mer guide strand (g-RNA), was complexed/annealed to yield the three P^{M}, P^{T} and P^{N}-escorted duplex siRNA conjugates (P^{x}Ep-siRNAs; where x=M, T or N).

As illustrated in FIG. 1E, to confirm the presence of both strands and integrity of the complex, we visualized the annealed, polymer conjugates by staining with ethidium bromide (EtBr). The polymer alone was not stained by EtBr, as exemplified by polymer-P^{N} (lane P^{N}), whereas the siRNA duplex was stained, and when conjugated to the polymer escorts, displayed a retarded migration through the gel as a result of increased size. A non-denaturing polyacrylamide gel with Tris borate buffer (pH 8.5) that is well above the pKa of PDMAEMA (˜pH 7.4) ensured that even the P^{N}Ep-RNA entered the gel. No such retarded mobility was observed when the siRNA duplexes were simply mixed but not conjugated to the polymers, indicating that polyplex formation was unlikely to occur. When conjugated in the PEp-siRNAs, the shift in the visualized siRNA as a result of higher molecular weight was uniform. The higher band for each of the PEp-siRNAs indicated that the flanking polymer escorts were bis-conjugated and homogenous, rather than a mixture of mono- and bis-conjugated RNA. Further, no free siRNA band was observed, indicating that the click-conjugation reaction was efficient and that high purity conjugates were prepared.

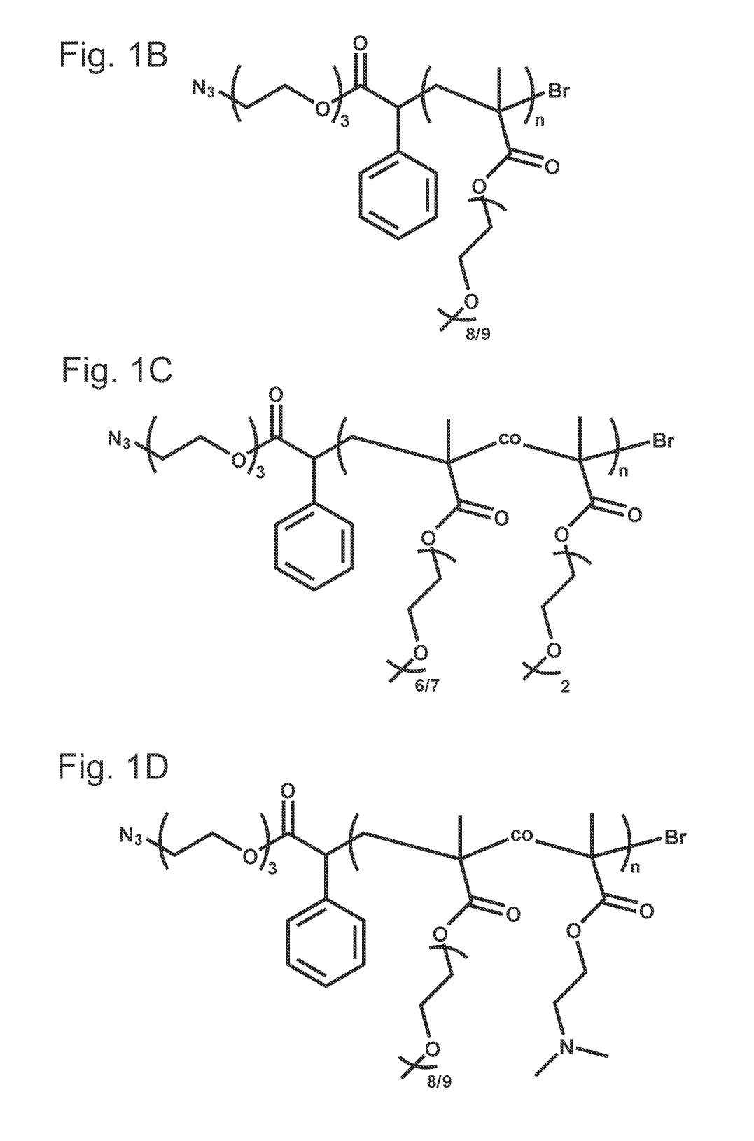

The representative siRNA delivery systems hereof, which include RNA-polymer conjugates, exhibit both nuclease resistance and auto-transfectability yielding a stand-alone siRNA delivery vehicle. The activity of the siRNA delivery systems hereof can be directly compared to duplex transfection with a traditional polyplex forming reagent (for example, FUGENE®-HD available from Promega Corporation of Madison, Wis.) that provides the current standard. To determine whether resistance to exonuclease is conferred to the g-RNA strand that is simply hybridized within the p-RNA-polymer conjugate, we incubated the three PEp-siRNAs with ribonuclease A (RNaseA) that can rapidly degrade both single- and double-stranded RNA. We found that while siRNA (duplex) was almost completely degraded by RNaseA, all the PEp-siRNAs remained intact even after 2 hours (see FIG. 2). This result indicates that the escort architecture can be used to sequester and protect not only the directly conjugated p-RNA strand, but also the hybridized g-RNA sequence of the siRNA duplex from nuclease mediated degradation.

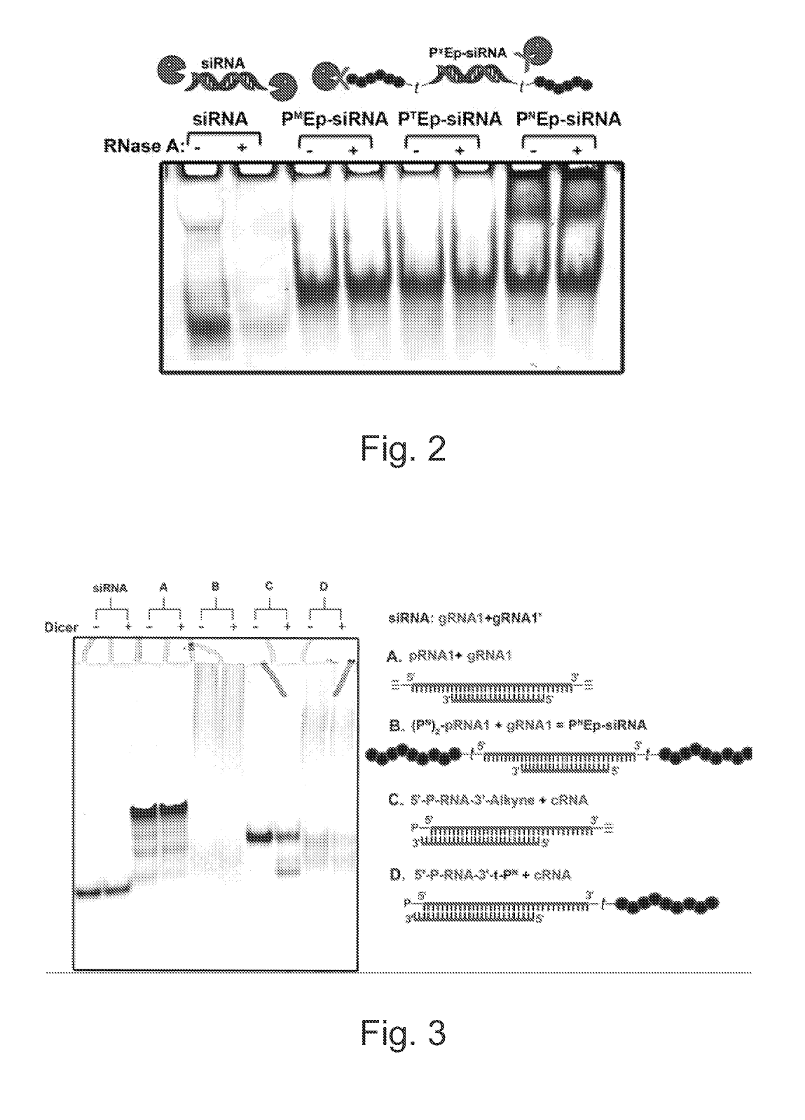

As illustrated in FIG. 3, the protective power of even just one covalent polymer escort also confers the PEp-siRNA with resistance to in vitro processing by the endonuclease dicer. Dicer processing is required for long RNA duplexes into canonical 21-mer duplexes with overhangs and helps their loading into RISC. However, dicer processing is not required for cleavage of the target mRNA. Cleavage of the target mRNA was mediated by argonaute for which the 21-mer g-RNA within the PEp-siRNA would be suitable and sufficient—if the g-RNA was accessible to RISC loading.

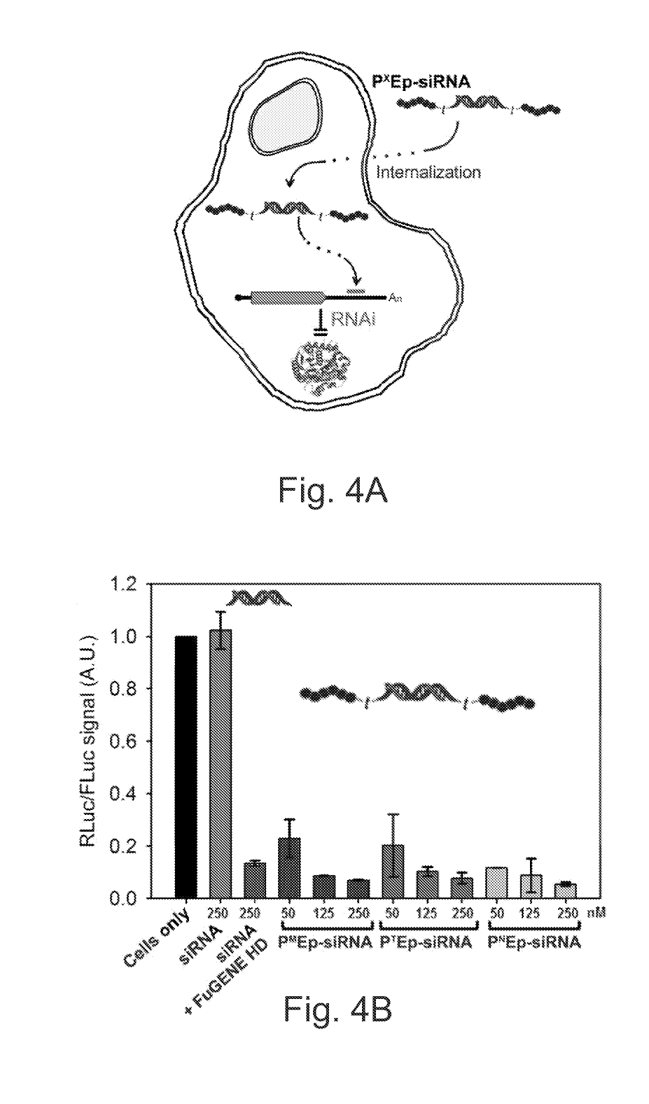

While the PEp-siRNAs were stable to RNaseA and dicer in vitro, the dissociation of the g-RNA from the PEp-siRNA is necessary in vivo for entry into RISC to induce an RNAi response. We therefore studied the efficiency of the PEp-siRNA conjugates in RNAi mediated knockdown of a target mRNA in cells. Drosophila S2 cells transfected with firefly and Renilla luciferase plasmids allow for the evaluation of RNAi-mediated knockdown in a dual luciferase assay. To assess the PEp-siRNAs, the hybridized g-RNA was designed to be complementary to a sequence in the 3′-untranslated region (3′-UTR) of the target mRNA from the Renilla luciferase (RLuc) gene. A schematic illustration of internalization and RNAi induction is provided in FIG. 4A. FIG. 4B illustrates a graph of relative Renilla luciferase (Rluc) signal. Following transfection of reporter plasmids, S2 cells were treated with 30 pmol siRNA without (−) or with (+) FUGENE HD for transfection or 50, 125 or 250 nM P^{x}Ep-siRNAs. The RLuc activity was determined after 24 hours incubation and normalized to the internal Fluc control. The ratio is reported relative to a control well without interfering RNA (cells only). Error bars represent the standard deviation from three separate experiments. As the mRNA target was in the 3′-UTR and not within the protein coding sequence, any knockdown was not due to blocked translation, but rather as a result of RNAi in this standardized assay. The firefly luciferase (FLuc) provides an internal control for transfection efficiency and protein production against which the knockdown of the RLuc signal can be compared. Following an initial transfection of the FLuc and RLuc reporter plasmids with FUGENE HD, a control duplex siRNA (30 pmol) was transfected after three hours, using an additional amount of FUGENE HD. This resulted in the knockdown of the RLuc signal measured after 24 hours. As illustrated in FIG. 4B, in the absence of the additional FUGENE HD, the effect of the control siRNA was negligible, indicating that after the initial transfection of the plasmids, no residual FUGENE HD remained and little non-specific internalization of siRNA occurred. In stark contrast, all three PEp-siRNAs required no transfection reagent and resulted in effective knockdowns. The PEp-siRNAs were each tested at 50, 125 and 250 nM concentrations (corresponding to 6, 15 and 30 pmoles of RNA, respectively) and evaluated 24 hours after addition. Each of the PEp-siRNAs resulted in greater knockdown activity than the equivalent or even half the amount of siRNA delivered through the transfection polyplex. Knockdown of the RLuc signal comparable to that with transfected siRNA could be achieved with just one-fifth the amount of RNA (6 pmol; 50 nM) in P^{N}Ep-siRNA that incorporates a positively charged DMAEMA in the copolymer segment (see FIG. 1D).

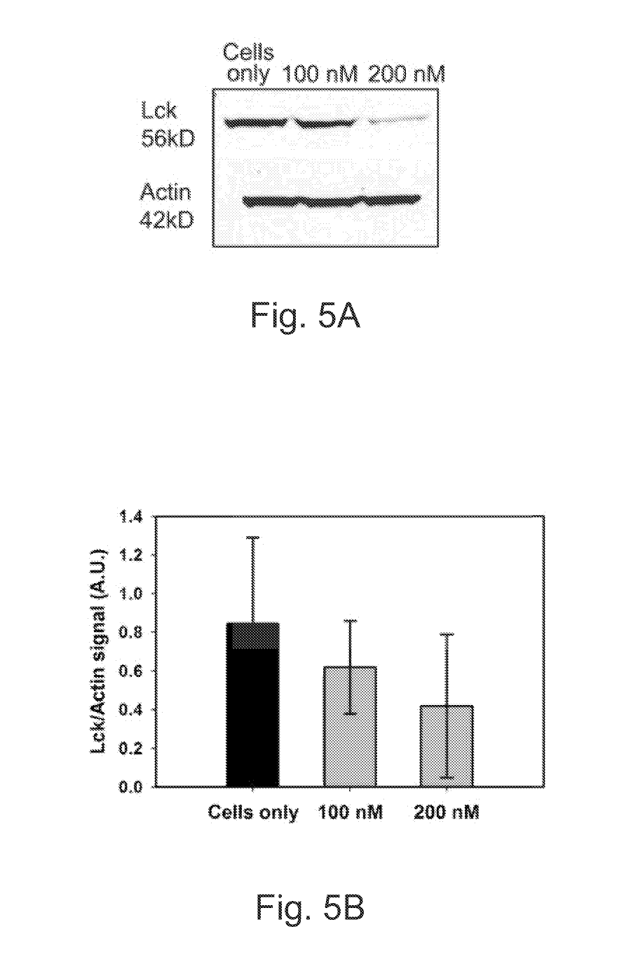

The success of the covalent polymer escorts for auto-internalization and release of the g-RNA that was effective in RNAi prompted tests of this architecture towards, for example, the knockdown of an endogenous gene in human embryonic kidney 293 (HEK293) cells. Lymphocyte-specific protein tyrosine kinase (Lck) is a member of the Src kinase family that is important in signal transduction events, particularly in T-cells. As the P^{N}Ep-siRNA was the most effective in the S2 cells, we used the Lck-P^{N}Ep-siRNA construct. This was simply added to the media with HEK293 cells. FIG. 5A illustrates a Western blot analysis of the inhibition of Lck in Hek293 cells using an auto-transfecting siRNA. Hek293 cells were plated (Cells Only) or transfected with 100 nM and 200 nM of Lck-P^{N}Ep-siRNA. Cells were cultured for 48 hours prior to lysis. Lysates were analyzed for total Lck and actin as a loading control. Compared to untreated cells, we observed specific and reproducible knockdown of Lck protein with the Lck-P^{N}Ep-siRNA without any transfection agent. In contrast, actin, serving as an internal control for gene expression, was unaffected as assayed by Western blotting. FIG. 5B sets forth a graph showing densitometric quantitation of Western blots with the Lck signal normalized to the actin control. Error bars represent standard deviation from three independent experiments. The quantitation of the relative protein expression levels indicates that the siRNA delivery systems hereof are viable in human cells to knockdown expression of an endogenous gene.

Given the viability of the auto-transfecting siRNA delivery systems in RNAi across different cell types, a variety architectures may, for example, be used to boost efficacy. For example, constructs may include a 5′-phosphate and other mimics through chemical modifications for added stability of the g-RNA strand while enhancing release from the duplex. Modifications may also be made to enhance oligonucleotide polymer synergy. Modifications to one or more residues of the oligonucleotide may be as simple as incorporation of 2′-deoxy residues (as in DNA) or other sugar or phosphate backbone modifications as in PNA. Nucleobase modifications that affect hybridization of the complexing agent/strand to the bioactive strand may also be incorporated to enhance complexation and/or release of the bioactive strand.

Studies hereof demonstrate that the representative PEp-siRNA “escort” conjugate architectures or oligonucleotide delivery systems hereof (with a single polymer or flanking polymers) provide a robust bioactive agent in one embodiment that induces RNA interference gene knockdown. These representative PEp-siRNA hybrids and other complexing agent-polymer conjugates hereof are obtained readily and efficiently by, for example, a simple post-synthetic click reaction, filtration and complexing (via, for example, annealing). The complexing agent-polymer conjugate (for example, PEp-siRNA) architecture simultaneously confers nuclease resistance and cell permeability to the complexed RNA/DNA. While non-specific polyplexes with RNA or certain disulphide linked polymer or nanostructure siRNA conjugates in the reducing cellular environment release the siRNA duplex, in a number of embodiments hereof, the polymer escorts/conjugates remain covalently conjugated via, for example, a triazole linkage to the complexing agent (for example, a passenger strand of RNA). Thus, rather than releasing the entire complex/duplex (for example, an siRNA duplex) once internalized, the delivery systems hereof (for example, PEp-siRNA) retain the ability to deliver only the hybridized strand of RNA/DNA/PNA/chimera as the payload (for example, for effective RNA silencing). This has significant implications for techniques such as RNAi, as it simplifies the design and could avoid off-target effects that may arise from the complexing agent (for example, a passenger strand of siRNA). The architectures hereof, which use one or more polymer escorts, is highly amenable to customization and inclusion of other polymer associated moieties for multi-modal delivery of therapeutic agents.

The representative examples provided herein set forth designs providing exemplification of a robust method for bio-conjugation of a nuclease resistant auto-transfecting siRNA. Once again, other conjugate architectures can be prepared, and targeting agents may be readily tethered to the conjugates using incorporated or inherent functionalities of the polymers. Because, in a number of embodiments, the polymer segments for the conjugates are prepared by a controlled polymerization procedure one skilled in the art appreciates that the ability to incorporate site specific functionality into the tetherable polymer enables preparation of conjugates with many well defined predictable architectures.

In the representative examples hereof, ATRP was used to prepare the α-functional polymer for clicking to the passenger RNA. ATRP and other CRP may also, for example, provide an co-chain end functionality (or other site-specific functionality) that can be used to incorporate targeting agents and/or other moieties. Furthermore since a small molecule ATRP initiator may be designed to incorporate more than one residual functionality into the initiator, it is straightforward to incorporate a degradable link (for example, an ester linkage) that will allow the “escort” to be degraded after delivery of the complexed RNA or DNA (for example, siRNA).

Experimental

Materials.

Oligo(ethylene oxide)monomethyl ether methacrylate (average molecular mass ˜475, ˜300 and 188 g/mol, OEOMA₄₇₅, OEOMA₃₀₀, MEO₂MA respectively), Acetonitrile, ascorbic acid, CuBr₂ and tin(II) 2-ethylhexanoate were purchased from Aldrich in the highest available purity. Copper sulfate pentahydrate and sodium ascorbate were purchased from Alfa Aesar. Tris(2-pyridylmethyl)amine (TPMA) was purchased from ATRP Solutions. Standard RNA phosphoramidites with labile phenoxyacetyl (PAC) protecting group, 3′-O-propargyl guanosine CPG column and appropriate reagents for solid phase RNA synthesis were purchased from Chemgenes (Wilmington, Mass., USA). The 5′-hexynyl modifier phosphoramidite was purchased from Glen Research (Sterling, Va., USA). Monomers were passed over a column of basic alumina prior to use. N3-PEG3-BPA (ATRP initiator) was prepared as previously described.¹

Instrumentation.

Molecular weight and molecular weight distribution (M_{w}/Mₙ) were determined by GPC. The GPC system used a Waters 515 HPLC Pump and Waters 2414 Refractive Index Detector using PSS columns (Styrogel 10², 10³, 10⁵ Å) in dimethylformamide (DMF) as an eluent at a flow rate of 1 mL/min at 50° C. and in tetrahydrofuran (THF) as an eluent at a flow rate of 1 mL/min at 35° C. All samples were filtered over anhydrous magnesium sulfate and neutral alumina prior to analysis. The column system was calibrated with 12 linear poly(methyl methacrylate) standards (Mₙ=800˜1,820,000).

RNA Synthesis:

Solid phase synthesis of the RNA was performed on a Mermade-4 (Bioautomation, Plano, Tex., USA) automated synthesizer. Synthesis and deprotection of the RNA was performed with standard protocols following recommendations of the manufacturer. After deprotection, the RNA was purified using 20% denaturing polyacrylamide gel electrophoresis (with 8 M urea). The RNA band in the gel was excised and eluted overnight in TE_{0.1} buffer (10 mM Tris.HCl, 0.1 mM EDTA, pH 7.5). The eluted RNA was desalted using a C18 Sep-Pak cartridge (Waters, Milford, Mass., USA). Finally the RNA was characterized by matrix-assisted laser desorption/ionization (MALDI) mass spectroscopy using 3-hydroxypicolinic acid as matrix. Table 1 sets forth the sequence and chemical modifications of the oligonucleotides used in this study. The sequences are also set forth in the sequence listing at the end of the specification. The MALDI mass of the RNAs were used to confirm the successful synthesis of the RNA. (P indicates a 5′-phosphate group).

TABLE 1

| | | | | Mass | Mass |
|---|---|---|---|---|---|
| Name | Sequence | 5′-terminus | 3′-terminus | Calculated | Found |
| p-RNA (RLuc) | 5′-UGG CGG AGG UGG GUA | Phosphohexynyl | O-propargyl | 11268.4 | 11290.5 |
| seq. ID no. 1 | UCU GGA UGU GGU U GG CUC G-3′ | | | | (M + Na⁺) |
| g-RNA (RLuc) | 5′-CUC ACA UUU ACA UAU | OH | OH | 6558.9 | 6559.9 |
| seq. ID no. 2 | UCA CAG-3′ | | | | |
| g-RNA′ (RLuc) | 5′-G UGG GUA UCU GGA | OH | OH | 6454.8 | 6457.6 |
| seq. ID no. 3 | UGU GGU U-3′ | | | | |
| | (to make duplex siRNA) | | | | |
| p-RNA | 5′-UGU CAU AAG CCA UGC | Phosphohexynyl | O-propargyl | 10926.4 | 10927.5 |
| (Lck) | CUU CUG CAA UUU GCC | | | | |
| seq. ID no. 4 | UCG A-3′ | | | | |
| g-RNA (Lck) | 5′-CAA AUU GCA GAA GGC | OH | OH | 7057.9 | 7058.5 |
| seq. ID no. 5 | AUG GC dT dT-3′ | | | | |
| pRNA1 | 5′-AUG ACA UAA GGU GGA | Phosphohexynyl | O-propargyl | 11860.7 | 11864.2 |
| seq. ID no. 6 | AGC CGG GCA UAA CUU | | | | |
| | AGU AAA-3′ | | | | |
| gRNA1 | 5′-GUU AUG CCC GGC UUC | OH | OH | 6554.8 | 6558.2 |
| seq. ID no. 7 | CAC C dT dT-3′ | | | | |
| gRNA1' | 5′-GGU GGA AGC CGG GCA | OH | OH | 6784.0 | 6788.9 |
| seq. ID no. 8 | UAA C dT dT-3′ | | | | |
| 5′-P-RNA-3′- | 5′-P-AUG ACA UAA GGU GGA | Phosphate | O-Propargyl | 11780.6 | 11803.7 |
| alkyne | AGC CGG GCA UAA CUU | | | | (M + Na⁺) |
| seq. ID no. 9 | AGU AAA-3′ | | | | |
| cRNA | 5′-AUG CCC GGC UUC CAC | OH | OH | 8502.1 | 8503.0 |
| seq. ID no. 10 | CUU AUG UCA UAG-3′ | | | | |

Polymer Synthesis:

Bio-compatible conditions for an ATRP as disclosed in PCT International Patent Application No. PCT/US12/51855 were employed during the “click” conjugation of the selected azido polymer to the dialkyne carrier RNA. Monomer, N3-PEG3-BPA, and CuBr2/TPMA (a 10× catalyst solution was prepared in DMF and aliquot into the reaction mixture) were added to 50% w/v toluene in a 10 mL Schlenk flask. The flask was sealed and degassed by bubbling with nitrogen for 10 minutes. After degassing the reaction mixture a degased solution of tin(II) 2-ethylhexanoate was injected to generate Cu(I) and the reaction was heated at 60° C. for 1 h. The reaction was stopped by dilution with tetrahydrofuran and passing the reaction mixture over a short column of basic alumina followed by precipitation into ethyl ether. The polymer was dried overnight under vacuum and molecular weight was determined using GPC. P^{M}: Mₙ=21,000 M_{w}/Mₙ=1.13; P^{T}: Mₙ=20,500 M_{w}/Mₙ=1.05; P^{N}: Mₙ=26,000 M_{w}/Mₙ=1.09.

Click Conjugation to Obtain PEp-RNAs:

To ensure both the alkynes in the RNA reacted with the azide group on the polymer, a 20 fold excess of the polymer (500 μM) over the p-RNA (25 μM) was used in the click reaction. Click conjugation of the p-RNA to the polymers was performed in Tris buffer (20 mM, pH 7.5), CuSO₄ (250 μM), 0.6% v/v acetonitrile and sodium ascorbate (1 mM) in 75 μL final reaction volume. All the reactants except CuSO₄ were mixed and degassed by bubbling with argon for five minutes. The reaction was started by the addition of a degassed solution of CuSO₄ to the reaction mixture. The reaction was allowed to run for 1.5 hours and the resulting PEp-RNA was separated from unreacted starting materials using an Amicon Ultra-0.5 centrifugal filter device with a 30,000 molecular weight cutoff. The amount of RNA was quantitated by absorbance at 260 nm.

Annealing to Obtain siRNA and PEp-siRNAs:

Annealing was performed by heating equimolar g-RNA′ or PEp-RNAs and g-RNA at 60° C. for 5 min and allowing to cool to ambient room temperature (−25° C.).

Polyacrylamide Gel Electrophoresis of Duplex siRNA and Polymers:

To determine if specific or non-specific aggregation of siRNA to the polymers was occurring, polymers P^{M}, P^{T} and P^{N} (150 pmol) were combined with siRNA duplexes (150 pmol) in 1×PBS for 10 min and then loaded on a native 10% polyacrylamide gel and stained with EtBr. Alternately, the siRNA (150 pmol) and PEp-siRNAs (150 pmol) were loaded on a 10% polyacrylamide gel (Tris borate buffer; pH 8.5) and stained with EtBr (FIG. 1E). The gels show no non-specific aggregation between the polymers and the siRNA.

RNase a Stability of PEp-siRNAs.

P^{x}Ep-siRNA (with P^{M}, P^{T} and P^{N}) or siRNA duplex (150 pmol) were incubated with RNase A (OMEGA-7U) for 2 h at RT. and the reaction mixtures were loaded on a 10% native polyacrylamide gel and stained with EtBr. The gel shows degradation of the siRNA duplex, but no degradation of either conjugate is observed (FIG. 2).

Dicer Cleavage Study of the PEp-siRNA.

To study whether these PEp-siRNAs are substrate for Dicer, a Dicer cleavage study was performed with recombinant Dicer enzyme. 300 pmoles of the modified RNA was incubated with 2 units of human recombinant Dicer enzyme (Genlantis, San Diego, Calif.) in 1× Dicer reaction buffer (110 mM Tris.HCl, 40 mM HEPES (pH=7.6), 200 mM NaCl, 2.5 mM MgCl₂, 2 mM ATP, 20 μL final volume) for 12 hrs at 37° C. The reaction was then stopped with 5 μL of 5 mM EDTA and the 25 μL of native gel loading solution (40% glycerol, 100 mM Tris.HCl, pH=7.5 and 10 mM EDTA) was added to the reaction mixture. Finally the samples were loaded in a 10% non-denaturing polyacrylamide gel and visualized by EtBr staining. Dicer cleavage of the P^{N}Ep-siRNA and related constructs. While construct-C showed Dicer cleavage (see FIG. 3), all other RNAs did not get cleaved by Dicer. This shows that the P^{N}Ep-siRNAs are not substrate for Dicer enzyme to release duplex siRNA which indicates that they will work in RNAi in a Dicer independent pathway plausibly by releasing the guide strand from the conjugate. The sequence and mass of the RNAs are set forth, for example, in Table 1.

Dual Luciferase Assay for RNAi in Drosophila S2 Cells.

Drosophila S2 cells (100 μl of 200,000 cells per mL) were plated on a 96-well plate in Schneider's media. On a separate tube, Firefly reporter plasmid (pGL3, Promega-20 ng, available from Promega Corporation of Madison, Wis.) and Renilla reporter plasmid (CJ22, Addgene-40 ng) were added to 98 μl of Schneider's media to a final volume of 100 μl. To this solution, 6 μl of FuGENE HD reagent was added and mixed by pipetting the solution up and down with pipettor. Following a 10 min incubation, 10 μl of this solution was added per well. The 96-well plate was incubated for 3 h for the reporter transfection to take place. Following the 3 h incubation, The PEp-siRNA against the Renilla reporter (6, 15 or 30 pmol) in 10 μl of 1×PBS added per well, respectively. In the control reactions, the siRNA duplexes (30 pmol) were mixed with either an additional 0.6 μl of FuGENE HD or 0.6 μl of 1×PBS in 10 μl of 1×PBS for 10 minutes and the solution was added to the well. The plates were incubated for 6 h and then 10 μl of 5.5 mM CuSO₄ was added per well to induce expression of the reporter genes.

After 24 h, since initial transfection of the reporter plasmids, 20 μl of 1× Passive Lysis Buffer (PLB) was added to each well and the plate was shaken on a plate rocker for 15 min. Following lysis, the luciferase activity of each well was read on a TECAN M-1000 with the Dual Luciferase® protocol using 100 μl dispense volumes for each reagent with 2 s delay for a 10 s integration read time.

Culture and Transfection of HEK293 Cells.

The HEK293 cell line was purchased from ATCC (American Tissue Culture Collection). HEK293 cells were maintained in 1×MEM (Gibco) supplemented with 10% fetal bovine serum, 1×L-glutamine (Gibco) and 1×MEM non-essential amino acids (Gibco), and were passaged as suggested by the manufacturer. One day prior to transfection, HEK293 cells were moved to 6 well plates. At 50% confluency, approximately 24 h after plating, 100 nM to 200 nM of Lck-P^{N}Ep-siRNA was added directly to the media in the 6 well plates. The expression level of Lck in the HEK293 cells was determined 48 h after transfection by Western blot analysis.

Western Blot Analysis for Lck.

Hek293 cultures were lysed and collected in 1×SDS lysis buffer containing protease and phosphatase inhibitors (Sigma). Lysates were sonicated and debris was collected by centrifugation at 14,000 g for 10 min at room temperature. The supernatant was collected and stored at −80° C. Total protein lysates were quantified by Micro Bicinchoninic Acid (BCA) (Pierce) colorimetric protein assay. For analysis, 50 μg of each sample was prepared in 1×LDS buffer containing a reducing agent, boiled for 5 minutes at 95° C., separated using a NuPAGE 4-12% Bis-Tris gel (Life Technologies), and transferred onto a nitrocellulose membrane. Membranes were blocked for 1 h at room temperature in 1×TBS-Tween 20 (TBS-T) with 5% milk and incubated in the primary antibody D88 against Lck (Cell Signaling) overnight in 1×TBS-T with 5% BSA at 4° C. with gentle agitation. Membranes were washed with 1×TBS-T and incubated at room temperature for 1 h in HRP conjugated Anti-Rabbit secondary antibody (Cell Signaling) in 1×TBS-T with 5% milk. West Pico ECL (Pierce) was used for signal detection. Membranes were stripped in stripping buffer PLUS (Pierce), washed in 1×TBS-T, and blocked in 1×TBS-T with 5% milk for 30 minutes at room temperature. Membranes were incubated with a β-Actin primary antibody (Sigma) in 1×TBS-T with 5% milk at room temperature for 45 min, washed, and incubated in HRP conjugated Anti-Mouse secondary for 1 hr at room temperature in 1×TBS-T with 5% milk. Signal was detected with West Pico ECL (Pierce), and results were quantitated using densitometry.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A delivery system for delivering a biologically active agent, comprising:
a conjugate comprising a complexing agent comprising an oligonucleotide, a peptide nucleic acid or chimera thereof and a polymer covalently attached to the complexing agent, the complexing agent of the conjugate being adapted to complex a biologically active agent thereto after formation of the conjugate.

2. The delivery system of claim 1 wherein the biologically active agent is a partially or fully complementary strand of RNA, DNA, PNA or chimera.

3. The delivery system of claim 1 wherein the biologically active agent is a partially or fully complementary strand of guide RNA, and the partially or fully complementary strand of guide RNA is adapted to effect RNA interference.

4. The delivery system of claim 3 wherein the complexing agent comprises a passenger strand of RNA.

5. The delivery system of claim 1 wherein the conjugate is prepared by reacting a functional group on the polymer with a functional group on the oligonucleotide, peptide nucleic acid or chimera.

6. The delivery system of claim 5 wherein the reaction of the functional group on the polymer to with the functional group on the oligonucleotide is a “click” reaction.

7. The delivery system of claim 5 wherein the reaction of the functional group on the polymer to with the functional group on the oligonucleotide is a Staudinger ligation, an azide-alkyne cycloaddition, a reaction of tetrazine with a trans-cyclooctene, a disulfide linking reaction, a thiol ene reaction, a hydrazine-aldehyde reaction, a hydrazine-ketone reaction, a hydroxyl amine-aldehyde reaction, a hydroxyl amine-ketone reaction or a Diels-Alder reaction.

8. The delivery system of claim 1 wherein the polymer is formed via controlled radical polymerization.

9. (canceled)

10. The delivery system of claim 1 wherein the polymer has a molecular weight between 1 kDa and 60 kDa.

11. (canceled)

12. The delivery system of claim 1 wherein the polymer has a polydispersity between 1 and 2.

13.-14. (canceled)

15. The delivery system of claim 1 wherein the polymer is a polyacrylate, a polymethacrylate, a polyacrylamide, a polymethacrylamide, a polystyrene, a polyethylene oxide, a poly(organo)phosphazene, a poly-1-lysine, a polyethyleneimine, a poly-d,l-lactide-co-glycolide, or a poly(alkylcyanoacrylate).

16. The delivery system of claim 12 wherein the polymer is formed via controlled radical polymerization or activator generated by electron transfer atom transfer radical polymerization.

17. The delivery system of claim 16 wherein the polymer is formed via atom transfer radical polymerization.

18. The delivery system of claim 1 further comprising at least a second polymer attached to the complexing agent.

19. The delivery system of claim 1 wherein the polymer includes at least one of a targeting agent group or a group that is cationic under physiological conditions such that the conjugate is auto-transfecting.

20. The delivery system of claim 1 wherein the biologically active agent separates from the conjugate in vivo.

21. A method of synthesizing a delivery system for delivering a biologically active agent, comprising:
preparing a conjugate by reacting a polymer with complexing agent to covalently attach the polymer to the complexing agent, the complexing agent comprising an oligonucleotide, a peptide nucleic acid or chimera thereof, the conjugate being adapted to complex a biologically active agent thereto after preparation of the conjugate; and
complexing the biologically active agent to the conjugate.

22.-40. (canceled)

41. An auto-transfecting system, comprising:
a conjugate comprising a complexing agent comprising an oligonucleotide, a peptide nucleic acid or chimera and a polymer covalently attached to the complexing agent, the polymer having a molecular weight distribution less than 2 and a molecular weight between 1 and 60 kDa, the polymer further comprising at least one of a targeting agent group or a group that is cationic under physiological conditions.
a biologically active agent complexed to the complexing agent of the conjugate after formation of the conjugate.

42. The system of claim 41 wherein the biologically active agent is a partially or fully complementary strand of RNA, DNA, PNA or chimera thereof.

43. The system of claim 42 wherein the biologically active agent is a partially or fully complementary strand of guide RNA, and the partially or fully complementary strand of guide RNA is adapted to effect RNA interference.
